(19) United States
(12) Patent Application Publication
(10) Pub. No.: US 2004/0266875 A1
(43) Pub. Date: Dec. 30, 2004

(54) Method for treatment and/or prophylaxis

(76) Inventor: Neil Roland McGregor, Eleebana (AU)

Correspondence Address: SUGHRUE MION, PLLC, 2100 PENNSYLVANIA AVENUE, N.W., SUITE 800, WASHINGTON, DC, 20037, US

(21) Appl. No.: 10/483,394

(22) PCT Filed: Jul. 5, 2002
(86) PCT No.: PCT/AU02/00892

(30) Foreign Application Priority Data
Jul. 10, 2001 (AU) PR 6259

(51) Int. Cl. A61K 31/198

(52) U.S. Cl. 514/561

(57) ABSTRACT

The present invention relates generally to muscle cramp and/or muscle stiffness and to a method for treating same. More particularly, the present invention provides a method for treating and/or reducing the likelihood of developing cram and/or muscle stiffness in a subject and to compositions effective in the treatment and/or prophylaxis of muscle cramp and/or muscle stiffness.

### FIELD OF THE INVENTION

The present invention relates generally to muscle cramp and/or muscle stiffness and to a method for treating same. More particularly, the present invention provides a method for treating and/or reducing the likelihood of developing cramp and/or muscle stiffness in a subject and to compositions effective in the treatment and/or prophylaxis of muscle cramp and/or muscle stiffness.

### BACKGROUND OF THE INVENTION

Bibliographic details of the publications referred to by author in this specification are collected at the end of the description.

Reference to any prior art, in this specification is not, and should not be taken as an acknowledgment or any form of suggestion that this prior art is common general knowledge or forms a part of the common general knowledge in Australia or any other country.

Muscle cells may be attached to bones (skeletal muscle) and produce movements of the limb and trunk. They may be attached to skin such as, for example, those producing facial expression (smooth muscle) or they may enclose hollow cavities, such as those in the heart (cardiac muscle) or bladder. Skeletal muscle is caused to contract by innervation by motor neurones whereas smooth and cardiac muscle may be caused to contract by nerves, chemical messengers such as hormones and by spontaneously induced mechanisms.

Motor neurones make up the somatic division of the peripheral nervous system. These neurones run from the central nervous system (brain and spinal column) to skeletal muscle cells. Excitation of motor neurones leads to the contraction of skeletal muscle cells.

Muscle cramp or stiffness is associated with involuntary contractions of muscle cells and particularly skeletal muscle cells. A very common form of skeletal muscle cramp is exercise-associated muscle cramp, however cramp or muscle stiffness occurs naturally or as a symptom of a number of acquired or inherited disorders. Other forms of muscle cramp include: occupational cramp, including writers cramp and nocturnal cramps.

The cause of muscle cramp or stiffness is not well understood although various theories have been proposed. For example, it has been proposed that electrolyte imbalance leads to inappropriate muscle contraction which may be alleviated by the administration of salt. It has also been proposed that exercise-associated muscle cramp occurs when muscle fatigue causes an imbalance between excitatory and inhibitory nerve signals.

The prevalence of muscle cramp or stiffness in the general population is not known. In the particular case of exercise-associated muscle cramp, this is estimated to be the most common clinical problem encountered by medical staff who treat athletes at endurance events. In the case of subjects with muscular disorders, muscle cramp or stiffness can be a very painful symptom of the disorder.

Available treatments include “salt”, quinine and Vitamin B, however, the mode of action and efficacy of these treatments has not been fully established. Furthermore, significant unwanted side-effects may occur, depending on the dosage and condition of the subject being treated.

Accordingly, there is a need to understand the cause of muscle cramp or stiffness and to develop therapeutic and prophylactic strategies which selectively target the problem. In the work leading up the present invention, the inventor has developed a composition which effectively targets cramps such as nocturnal and exercise-associated muscle cramp or muscle stiffness.

### SUMMARY OF THE INVENTION

Throughout this specification, unless the context requires otherwise, the word “comprise”, and variations such as “comprises” and “comprising”, will be understood to imply the inclusion of a stated element or integer or step or group of elements or integers or steps but not the exclusion of any other element or integer or step or group of elements or integers or steps.

In a first aspect, the present invention provides a method for the treatment and/or prophylaxis of muscle cramps and/or muscle stiffness in a mammal comprising administering to said mammal an effective amount of an inhibitory neurotransmitter amino acid and/or a precursor thereof, or a functional derivative, chemical equivalent, mimetic, analogue or homologue thereof for a time and under conditions sufficient to reduce, inhibit or otherwise down-regulate the duration, severity and/or frequency of muscle cramp and/or muscle stiffness.

Another aspect of the present invention provides a method for the treatment and/or prophylaxis of skeletal muscle cramps and/or skeletal muscle stiffness in a mammal comprising administering to said mammal an effective amount of an inhibitory neurotransmitter amino acid and/or a precursor thereof, or a functional derivative, chemical equivalent, mimetic, analogue or homologue thereof for a time and under conditions sufficient to reduce, inhibit or otherwise down-regulate the duration, severity and/or frequency of skeletal muscle cramp and/or skeletal muscle stiffness.

Yet another aspect of the present invention is directed to a method for the treatment and/or prophylaxis of skeletal muscle cramps and/or skeletal muscle stiffness in a mammal comprising administering to said mammal an effective amount of glycine and/or a precursor thereof such as threonine, or a functional derivative, chemical equivalent, mimetic, analogue or homologue thereof for a time and under conditions sufficient to reduce, inhibit or otherwise down-regulate the duration, severity and/or frequency of skeletal muscle cramp and/or skeletal muscle stiffness.

Yet another aspect of the present invention is directed to a method for the treatment and/or prophylaxis of skeletal muscle cramps and/or skeletal muscle stiffness in a mammal comprising administering to said mammal an effective amount of glycine and/or threonine, or a functional derivative, chemical equivalent, mimetic, analogue or homologue thereof for a time and under conditions sufficient to reduce, inhibit or otherwise down-regulate the duration, severity and/or frequency of skeletal muscle cramp and/or skeletal muscle stiffness.

Still another aspect of the present invention provides a composition comprising an inhibitory neurotransmitter amino acid and/or a precursor thereof, or a functional derivative, chemical equivalent, mimetic, analogue or homologue thereof for use in the treatment and/or prophylaxis of muscle cramp and/or muscle stiffness in a mammalian subject.

Another aspect of the present invention provides a composition comprising an inhibitory neurotransmitter amino acid and/or a precursor thereof, or a functional derivative, chemical equivalent, mimetic, analogue or homologue thereof for use in the treatment and/or prophylaxis of skeletal muscle cramp and/or skeletal muscle stiffness in a mammalian subject.

Even yet another aspect of the present invention provides a composition comprising glycine and/or a precursor thereof such as threonine, or a functional derivative, chemical equivalent, mimetic, analogue or homologue thereof for use in the treatment and/or prophylaxis of skeletal muscle cramp and/or skeletal muscle stiffness in a mammalian subject.

In yet another aspect of the present invention there is provided a composition comprising glycine and/or threonine, or a functional derivative, chemical equivalent, mimetic, analogue or homologue thereof for use in the treatment and/or prophylaxis of skeletal muscle cramp and/or skeletal muscle stiffness in a mammalian subject.

Still yet another aspect of the present invention provide a composition comprising:

i) glycine; and/or

ii) threonine;

optionally together with at least one of;

iii) a chlorine anion

iv) a phosphate anion

v) a bicarbonate anion

for use in the treatment and/or prophylaxis of muscle cramp and/or muscle stiffness in a mammalian subject.

Even yet another related aspect of the present invention contemplates the use of a composition comprising:

i) glycine; and/or

ii) threonine;

optionally together with at least one of;

iii) a chlorine anion

iv) a phosphate anion

v) a bicarbonate anion

in the manufacture of a medicament for treatment and/or prophylaxis of muscle cramp and/or muscle stiffness in a mammalian subject.

### BRIEF DESCRIPTION OF THE DRAWINGS

FIG. 1 is a diagrammatic representation of the motor nerve supply to non-cranial nerves. Two inhibitory neurones synapse with the α-Motor Neurone. The signals for these two inhibitory neurones come from a) the Golgi tendon organ and b) the muscle spindle. The inhibitory neurone neurotransmitter is Glycine. The ∅ sign indicates the glycine synapses.

### DETAILED DESCRIPTION OF THE INVENTION

The present invention is predicated, in part, on the observation that muscle cramps in a subject can be alleviated by the oral administration of a composition comprising an inhibitory neurotransmitter amino acid.

Accordingly, one aspect of the present invention contemplates a method for the treatment and/or prophylaxis of muscle cramps and/or muscle stiffness in a mammal comprising administering to said mammal an effective amount of an inhibitory neurotransmitter amino acid and/or a precursor thereof, or a functional derivative, chemical equivalent, mimetic, analogue or homologue thereof for a time and under conditions sufficient to reduce, inhibit or otherwise down-regulate the duration, severity and/or frequency of muscle cramp and/or muscle stiffness.

More particularly, the present invention contemplates a method for the treatment and/or prophylaxis of skeletal muscle cramps and/or skeletal muscle stiffness in a mammal comprising administering to said mammal an effective amount of an inhibitory neurotransmitter amino acid and/or a precursor thereof, or a functional derivative, chemical equivalent, mimetic, analogue or homologue thereof for a time and under conditions sufficient to reduce, inhibit or otherwise down-regulate the duration, severity and/or frequency of skeletal muscle cramp and/or skeletal muscle stiffness.

Reference herein to an “neurotransmitter” is to be understood as a reference to a molecule which directly or indirectly modulates the permeability of an ion channel affecting the membrane potential of a muscle cell.

Reference herein to an “inhibitory neurotransmitter” means a molecule which directly or indirectly reduces the likelihood that a muscle cell will generate an action potential. Accordingly, the term encompasses “neuromodulators” as well as direct “neurotransmitters” of synaptic activity. Appropriate neurotransmitter agonists or antagonists or neurotransmitter receptor agonists and antagonists are also within the scope of the present invention and within the scope of the term “inhibitory neurotransmitter” used herein.

Examples of neurotransmitters are acetylcholine, histamine, endorphin, GABA (g-aminobutyric acid) and glycine.

Examples of inhibitory neurotransmitters are GABA and glycine.

The neurotransmitter of the present invention may be administered as an active or as an inactive precursor molecule. Threonine, which may be converted into glycine in vivo, is a particularly contemplated example of a precursor molecule.

Reference herein to “an inhibitory neurotransmitter amino acid” should be understood as a reference to an amino acid which functions as an inhibitory neurotranmitter.

Reference herein to “functional derivative, chemical equivalent, mimetic, analogue or homologue thereof” of an amino acid inhibitory neurotransmitter or of glycine or threonine should be understood to include reference to molecules from natural, synthetic or recombinant sources exhibiting at least one of the functional activities of an inhibitory neurotransmitter amino acid or of glycine or threonine and may be, for example, molecules obtained following natural-product screening. Derivatives include those made by chemical modification by addition or removal of one or more moieties.

Even still more particularly, the present invention contemplates a method for the treatment and/or prophylaxis of skeletal muscle cramps and/or skeletal muscle stiffness in a mammal comprising administering to said mammal an effective amount of glycine and/or a precursor thereof such as threonine, or a functional derivative, chemical equivalent, mimetic, analogue or homologue thereof for a time and under conditions sufficient to reduce, inhibit or otherwise down-regulate the duration, severity and/or frequency of skeletal muscle cramp and/or skeletal muscle stiffness.

Even more particularly, the present invention contemplates a method for the treatment and/or prophylaxis of skeletal muscle cramps and/or skeletal muscle stiffness in a mammal comprising administering to said mammal an effective amount of glycine and/or a precursor thereof such as threonine, or a functional derivative, chemical equivalent, mimetic, analogue or homologue thereof for a time and under conditions sufficient to reduce, inhibit or otherwise down-regulate the duration, severity and/or frequency of skeletal muscle cramp and/or skeletal muscle stiffness.

Even still more particularly, the present invention contemplates a method for the treatment and/or prophylaxis of skeletal muscle cramps and/or skeletal muscle stiffness in a mammal comprising administering to said mammal an effective amount of glycine and/or threonine, or a functional derivative, chemical equivalent, mimetic, analogue or homologue thereof for a time and under conditions sufficient to reduce, inhibit or otherwise down-regulate the duration, severity and/or frequency of skeletal muscle cramp and/or skeletal muscle stiffness.

In a particular embodiment of this aspect of the present invention, both glycine and threonine are administered.

Without limiting the present invention to any one theory or mode of action, it is thought that glycine enhances the ability of muscle cells to inhibit efferent alpha-motor neurone activity. Furthermore, it is thought that threonine crosses the blood brain barrier where it is converted to glycine to permit the inhibitory neurotransmitter activity of glycine within the central nervous system.

Reference herein to the treatment and/or prophylaxis of “muscle cramp and/or stiffness” should be understood as a reference to the symptoms of involuntary muscle contraction which are associated with a very wide range of conditions found in mammalian subjects. For example, muscle cramps and/or stiffness is found in exercise-induced muscle-fatigued subjects as well as in subjects suffering from inherited or acquired neurological, neuromuscular or muscular disorders or ataxia's, spasticity, dystonia; occupational, nocturnal or writers cramp.

Reference herein to “treatment” and “prophylaxis” is used in its broadest sense. The term “treatment” does not mean that muscle cramps or stiffness is totally cured. Similarly, “prophylaxis” does not mean that the subject will never develop cramps or muscle stiffness. Accordingly, these terms include amelioration of the condition including a reduced duration, severity, or frequency of muscle cramps or muscle stiffness.

The subject of treatment and/or prophylaxis herein is generally a mammal such as for example a human, primate, livestock animal (eg sheep, pig, cow, horse, donkey) companion animal (eg cat, dog) laboratory test animal (eg mouse, rabbit, rat, guinea pig, hamster) captive wild animal (eg fox, deer). Preferably, the mammal is a human or primate. Most preferably, the mammal is a human.

Another aspect of the present invention provides a composition comprising an inhibitory neurotransmitter amino acid and/or a precursor thereof, or a functional derivative, chemical equivalent, mimetic, analogue or homologue thereof for use in the treatment and/or prophylaxis of muscle cramp and/or muscle stiffness in a mammalian subject.

Another related aspect of the present invention provides a composition comprising an inhibitory neurotransmitter amino acid and/or a precursor thereof, or a functional derivative, chemical equivalent, mimetic, analogue or homologue thereof for use in the treatment and/or prophylaxis of skeletal muscle cramp and/or skeletal muscle stiffness in a mammalian subject.

Still another aspect of the present invention contemplates a composition comprising glycine and/or a precursor thereof such as threonine, or a functional derivative, chemical equivalent, mimetic, analogue or homologue thereof for use in the treatment and/or prophylaxis of muscle cramp and/or muscle stiffness in a mammalian subject.

Still yet another aspect of the present invention contemplates a composition comprising glycine and/or a precursor thereof such as threonine, or a functional derivative, chemical equivalent, mimetic, analogue or homologue thereof when used in the treatment and/or prophylaxis of muscle cramp and/or muscle stiffness in a mammalian subject.

Another aspect of the present invention contemplates a composition comprising glycine and/or threonine, or a functional derivative, chemical equivalent, mimetic, analogue or homologue thereof for use in the treatment and/or prophylaxis of skeletal muscle cramp and/or skeletal muscle stiffness in a mammalian subject.

Preferably, the composition comprises both glycine and threonine.

Administration of the composition of the present invention may be by any convenient route. Oral administration is generally preferred, although pharmaceutical forms of the present composition may be suitable for injectable use such as sterile aqueous solutions (where water soluble) and sterile powders for the extemporaneous preparation of sterile injectable solutions or dispersions. The composition must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof and vegetable oils. The proper fluidity can be maintained, for example, by the use of a coating such as lecithin. The prevention of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The compositions may be orally administered, for example, with an inert diluent or with an assimilable edible carrier, or it may be enclosed in hard or soft shell gelatin capsule, or it may be compressed into tablets, or it may be in powdered form or incorporated directly with the food of the diet. For oral therapeutic and/or prophylactic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.01 μg and about 2000 mg of active compound. Alternative amounts include between about 1.0 μg and about 1500 μg, between about 1 μg and about 1000 mg and between about 10 μg and about 500 mg.

The tablets, troches, pills, capsules and the like may also contain the components as listed hereafter: A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of particular conditions in living subjects.

The principal active ingredient or ingredients are compounded for convenient and effective administration in effective amounts with a suitable pharmaceutically acceptable carrier in dosage unit form. A unit dosage form can, for example, contain the principal active compounds in amounts ranging from 0.01 μg to about 70 g/100 grams. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients. Alternatively, amounts administered may be represented in terms of amounts/kg body weight. In this case, amounts range from about 0.001 μg to about 1000 mg/kg body weight may be administered. Preferred ranges include from about 50 μg to 500 mg 1 kg body weight 500 mg/kg body weight or about 0.01 μg to about or above 0.1 μg to about 250 mg/kg body weight are contemplated by the present invention.

In a related aspect, the present invention provides a composition comprising:

i) glycine; and/or

ii) threonine;

optionally together with at least one of;

iii) a chlorine anion

iv) a phosphate anion

v) a bicarbonate anion

for use in the treatment and/or prophylaxis of muscle cramp and/or muscle stiffness in a mammalian subject.

Even yet another related aspect of the present invention contemplates the use of a composition comprising:

i) glycine; and/or

ii) threonine;

optionally together with at least one of;

iii) a chlorine anion

iv) a phosphate anion

v) a bicarbonate anion

in the manufacture of a medicament for treatment and/or prophylaxis of muscle cramp and/or muscle stiffness in a mammalian subject.

The present invention is now further described with reference to the following non-limiting examples.

### EXAMPLE 1

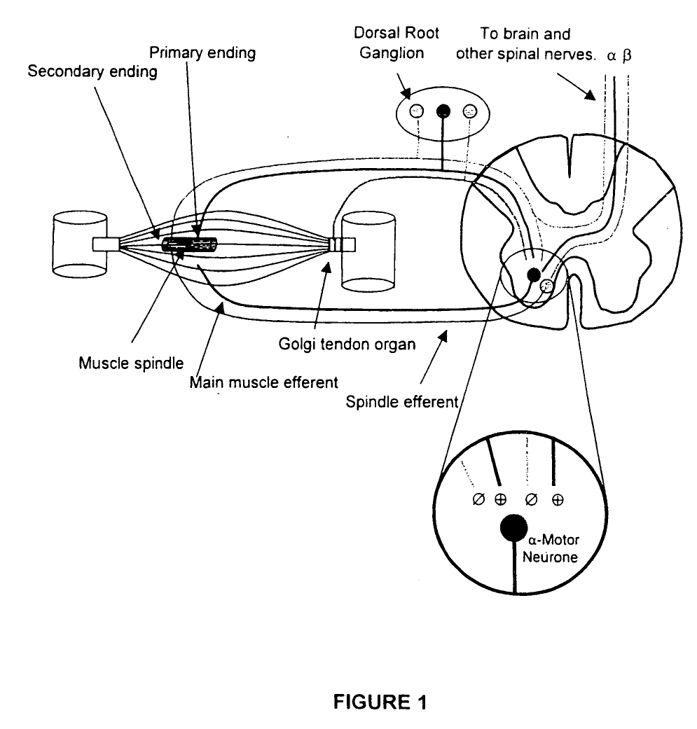

FIG. 1 shows the motor nerve supply to most non-cranial nerve muscles. The central signal comes from the brain via the α-motor neurone with secondary signals to the muscle spindle from the β nerve route. The α-motor neurone initiates the contraction of the muscle whilst the muscle spindle and the golgi tendon organ provide negative or inhibitory feedback to the α-motor neurone to inhibit the neurone. The neurotransmitter that controls the inhibitory response is the amino acid glycine. Glycine acts on the α-motor neurone by regulating the resting membrane potential. The glycine signal causes a change in chloride ion pumping which results in this change in resting potential. Falls in the availability of glycine in the central nervous system will therefore resulting a reduction of the ability to inhibit the α-motor neurone. There are several methods by which changes in this system can effect the development of cramps:

1) The reduction in glycine availability. A low level of threonine and or high level of serine will be able to reduce glycine availability in the central nervous system, the neurones and their synapses. Increased glycine conjugation in the liver through the glycine conjugation mechanisms will also reduce the available glycine and threonine (Kasuya et al., 1996).

2) Threonine transport disorders. A low level of threonine and or high level of serine will be able to reduce glycine uptake into the central nervous system or into the neurones and their synapses. Threonine availability is important as it is transported across the blood brain barrier where as glycine is not. Once threonine is transported it may then be converted to glycine for use in the CNS.

3) Electrolyte problems. Sodium and chloride availability are significantly influenced by the amount of fluid and electrolyte replacement associated with exercise—particularly in competitive sport. These alterations may also be seen in various disease conditions where electrolyte availability is influenced by the disease process.

4) Fall in high energy phosphate levels. This is usually associated with high exercise periods in sports people but may also occur in certain disease states.

### EXAMPLE 2

The following composition was tested in subjects:

| Compound | mg per gram |
|---|---|
| L-Glycine | 350 mg |
| L-Threonine | 150 mg |
| Sodium chloride | 250 mg |
| Sodium phosphate | 125 mg |
| Sodium bicarbonate | 125 mg |

### EXAMPLE 3

A 37 year old male had nocturnal muscle cramps for over 15 years and had tried all available drugs and remedies to correct the problem. One single 300 mg dose of the composition exemplified in Example 2 prevented the development of cramps during the night for the first time in all those years. The patient was monitored on a regular basis over 2 years. The subject takes a small amount of the composition exemplified in Example 2 every 1-2 weeks and has not developed nocturnal muscle cramps since the time of the first dose.

### EXAMPLE 4

A 27 year old sporting person who developed cramps regularly during sports events (cricket) took a 300 mg dose the supplement of Example 2 before every cricket game and has not developed cramps since that time whilst playing cricket over a summer of sport.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

Bibliography

Kasuya F, Igarashi K, Fukui M. Participation of a medium chain acyl-CoA synthetase in glycine conjugation of the benzoic acid derivatives with the electron-donating groups. Biochem Pharmacol; 51:805-809, 1996.

What is claimed is:

1. A method for the treatment and/or prophylaxis of muscle cramps and/or muscle stiffness in a mammal comprising administering to said mammal an effective amount of an inhibitory neurotransmitter amino acid and/or a precursor thereof, or a functional derivative, chemical equivalent, mimetic, analogue or homologue thereof for a time and under conditions sufficient to reduce, inhibit or otherwise down-regulate the duration, severity and/or frequency of muscle cramps and/or muscle stiffness.

2. A method according to claim 1 wherein said muscle cramps and/or muscle stiffness are skeletal muscle cramps and/or skeletal muscle stiffness.

3. A method according to claim 1 wherein said inhibitory neurotransmitter amino acid is glycine.

4. A method according to claim 3 wherein said precursor is threonine.

5. A method according to claim 4 wherein said composition comprises glycine and threonine.

6. A method according to claim 1 wherein said mammalian subject is a human subject.

7. A composition comprising an inhibitory neurotransmitter amino acid and/or a precursor thereof, or a functional derivative, chemical equivalent, mimetic, analogue or homologue thereof for use in the treatment and/or prophylaxis of muscle cramp and/or muscle stiffness in a mammalian subject.

8. A composition according to claim 7 wherein said neurotransmitter amino acid is glycine.

9. A composition according to claim 7 wherein said precursor is threonine.

10. A composition according to claim 9 wherein said composition comprises,
i) glycine; and/or
ii) threonine;
optionally together with at least one of;
iii) a chlorine anion
iv) a phosphate anion
v) a bicarbonate anion

11. A composition according to claim 10 where said composition comprises glycine and threonine.

12. Use of a composition comprising an inhibitory neurotransmitter amino acid and/or a precursor thereof, or a functional derivative, chemical equivalent, mimetic, analogue or homologue thereof in the manufacture of a medicament for treatment and/or prophylaxis of muscle cramps and/or muscle stiffness in a mammalian subject.

13. Use of a composition comprising;
i) glycine; and/or
ii) threonine;
optionally together with at least one of;
iii) a chlorine anion
iv) a phosphate anion
v) a bicarbonate anion
in the manufacture of a medicament for treatment and/or prophylaxis of muscle cramp and/or muscle stiffness in a mammalian subject.

14. A use according to claim 13 wherein said composition comprises glycine and threonine.
